(19) United States
(12) Patent Application Publication
(10) Pub. No.: US 2017/0296649 A1
(43) Pub. Date: Oct. 19, 2017

(54) METHOD FOR INDUCING T CELLS FOR CELL-BASED IMMUNOTHERAPY

(71) Applicant: Hiroshi KAWAMOTO, Kyoto (JP)

(72) Inventors: Hiroshi Kawamoto, Kyoto (JP); Kyoko Masuda, Kyoto (JP); Takuya Maeda, Kyoto (JP); Seiji Nagano, Kyoto (JP); Yoshimoto Katsura, Abiko (JP)

(21) Appl. No.: 15/326,940

(22) PCT Filed: Jul. 17, 2015
(86) PCT No.: PCT/JP2015/070622

§ 371 Date: Jun. 19, 2017

Related U.S. Application Data
(60) Provisional application No. 62/026,322, filed on Jul. 18, 2014.
(60) Provisional application No. 62/026,328, filed on Jul. 18, 2014.

(51) Int. Cl. A61K 39/245; C12N 7/00; A61K 39/00

(52) U.S. Cl. CPC A61K 39/245; C12N 7/00; A61K 2039/5158; A61K 2039/585; C12N 2710/16234

(57) ABSTRACT

Provided is a method for inducing T cells for a cell-based immunotherapy, which comprises the steps of: (1) providing human pluripotent stem cells bearing a T cell receptor specific for a desired antigen, and (2) inducing T cell progenitors or mature T cells from the pluripotent stem cells of step (1). Especially, a method for inducing T cells for a cell-based immunotherapy from cells of a person who is not the subject to be treated by the cell-based immunotherapy. The method provided herein may further comprise a step of co-culturing the T cell progenitors or mature T cells induced from the pluripotent stem cells with the lymphocytes of the subject to be treated by the cell based immunotherapy to verify that the T cells are not allogenicaly reactive against the subject.

The present application relates to a method for inducing T cells for cell-based immunotherapy. Specifically, a method for inducing T cells used for the cell-based immunotherapy in which allogenic T cells having a desired antigen specificity are transplanted to a subject to be treated.

### BACKGROUND ART

Each T cell expresses a T cell receptor (TCR) with different specificity. When an infectious disease develops, a T cell having a suitable specificity will proliferate to give a T cell population (clone) that will fight with the pathogen. This is the basic idea of the acquired immunity. If it is possible to artificially amplify a T cell with a given specificity, the amplified T cells may be used for the adoptive immunotherapy. The amplification of a specific T cell is called as “cloning”. In fact, autologous transplantation of antigen specific T cells prepared by amplifying the antigen specific T cell obtained from the patient has been clinically conducted. However, almost all autologous T cell transplantation therapies do not use a cell population purified to the extent of “cloned” cells. In addition, repeated in vitro sub-culturing of the cells might cause loss of the function to kill the cancer cells.

A method for providing T cells that are capable of infinitely proliferating by immortalizing the cells has been proposed. A cell may be immortalized and proliferated to give a cloned cell population. Procedures to immortalize a cell may include fusion of the cell with a cancer cell as well as long term culture of the cells with stimulating TCR under the presence of cytokines. However, auto-transplantation of thus obtained immortalized T cells may be dangerous. In addition, the cloning procedures could lower the cell function.

Cell-based immunotherapies in which T cells are transplanted proposed up to now are briefly explained.

### A. Cloning of T Cells Utilizing the Reprogramming Technique

Methods in which stem cells bearing genes encoding an antigen specific TCR are clonally expanded by using the reprogramming technique have been proposed. This method is expected to dissolve the problems in autologous transplantation of T cells. Specifically, pluripotent stem cells are generated from T cells by means of nuclear transplantation or the technique for establishing iPS cells. Patent applications directing to this concept have been submitted (WO2008/038579 and WO2011/096482). Papers on those methods have been published in 2010 and 2013:

1) Watarai H, A Rybouchkin, N Hongo, Y Nagata, S Sakata, E Sekine, N Dashtsoodol, T Tashiro, S-I Fujii, K Shimizu, K Mori, K. Masuda, H Kawamcto, H Koseki, and M Taniguchi. Generation of functional NKT cells in vitro from embryonic stem cells bearing rearranged invariant Vα14-Jα18 TCRα gene. Blood 115:230-237, 2010.
2) Vizcardo R, Masuda K, Yamada D, Ikawa T, Shimizu K, Fujii S-I, Koseki H, Kawamoto H. Regeneration of human tumor antigen-specific T cells from iPS cells derived from mature CD8+ T cells. Cell Stem Cell. 12: 31-36. 2013.

### 3) Nishimura T et al., Cell Stem Cell. 12: 114-226. 2013.

In those methods, ES cells or iPS cells are established from the patient's T cells, T cells are reproduced from those ES or iPS cells and then, the regenerated T cells are transplanted to the patient (autologous transplantation). However, the methods have at least three problems shown below: A1) iPS cells must be established from each patient and therefore, previous preparation for the therapy applicable for various people is impossible; A2) iPS cells are established for each patient and therefore, the quality and safety of the obtained iPS cells may vary each time; and A3) T cells differentiated from the T-iPS cells may become cancer.

B. T Cell Therapy in which T Cells Introduced with Genes Encoding a TCR are Used

Clinical test for gene therapies in which genes encoding an antigen specific T cell receptor (TCR) are isolated and the genes are transfected in the normal T cells obtained from the patient to be treated, the transfected T cells are then transplanted to the patient (autologous transplantation) have been conducted in various places (Morgan R. A. et al, Science, 314:126. 2006). The T cells are obtained as aggregate of various clones. According to this method, expression of TCRs originally present in the normal T cells are suppressed by, for example, siRNA (Okamoto S et al, Cancer Res 69:9003, 2009). Thus obtained T cells expressing only the specific TCR are subjected to the autologous transplantation. For example, genes encoding a T cell receptor specific for a WT1 antigen have neem isolated. Gene therapy using the TCR genes for treating WT1 expressing cancers has been conducted.

In the method B, T cells used for the therapy are also prepared from the T cells of the patient to be treated. This method has three problems as follows. B1) There is a risk that the patient's T cells become cancer, because this is a gene therapy; B2) The expression of endogenous genes encoding the original TCR in the T cells to be transplanted is not perfectly be suppressed and therefore, there is a risk of unintended reaction; B3) T cells must be prepared from each patient and therefore, previous preparation for the therapy applicable for various people is impossible

### C. Donor Lymphocyte Infusion

Bone marrow transplantation for hematological malignancy such as leukemia also has an aspect as a cell-based immunotherapy. That is, T cells contained in the transplanted bone marrow cells of the donor are expected to attack against the leukemia cells in the recipient. Donor lymphocyte infusion, in which donor's T cells are separately infused after the bone marrow transplantation in order to enhance the effect, has also been known. Recently, a new method in which clonally expanded T cells specific for a given antigen are infused has been proposed (Chapuis et al, Sci Transl Med, 5:174ra27, 2013).

In this method, the T cells to be infused are derived from a donor. However, the hematopoietic system of the recipient after receiving the bone marrow transplantation has become the same as that of the donor. Accordingly, the T cell infusion after the bone marrow transplantation is deemed as a sort of autologous transplantation. This method requires bone marrow transplantation and the patient needs to receive immunosuppressant for his/her entire life.

### D. Use of Umbilical Code Lymphocyte for Treating Another Person

Patients who received umbilical cord blood transplantation sometimes develop a viral infectious disease. In order to treat said patients, infusion of viral specific CTLs contained in umbilical cord blood derived from a person other than the person from whom the original umbilical cord was obtained has been proposed (Blood, 116: 5045, 2010). A patent application on an idea of transplanting CTLs of a donor having HLAs that match the patient's HLAs to the some extent but not completely has been submitted (WO2011/021503). However, T cells in the umbilical cord blood are aggregate of clones, i.e. an aggregate of the cells bearing a number of different TCRs. Therefore, cannot perfectly avoid a risk of exerting graft-versus-host disease (GVHD).

As discussed above, a variety of cell-based immunotherapies using T cells have been proposed. All therapies except D are autologous cell transplantation or are deemed to be autologous transplantation. Heterologous T cell transplantation is contrary to the common general technical knowledge. In the treatment of hematological malignancy such as leukemia, for example, bone marrow transplantation in which hematopoietic stem cells is, in general, conducted. In order to avoid the rejection of the donor's bone marrow by the recipient, bone marrow from a donor who has HLAs that match the recipient's HLAs is used. However, amino acid sequences of various proteins other than HLAs do not match between two people and donor's T cells may recognize those mismatches as targets for attack. As a result, a part of the transplanted donor's T cells attack against the recipient's body, i.e. graft-versus-host reaction could exert, and put the recipient to die (Ito et al Lancet, 331: 413, 1988).

A project to create a highly versatile iPS cell bank with donors having HLA haplorypes that are frequently found in Japanese people in homozygous is in progress. (CURANOSKI, Nature vol. 488, 139, 2012). However, in T cell transplantation, even if the donor has HLAs that completely match the recipient's HLAs, there is still a risk of graft-versus-host reaction. Further, when HLAs mismatch, more severe graft-versus-host reaction is expected. Accordingly, this iPS stock project has been inapplicable for the cell-based immunotherapy that uses T cells.

### PRIOR ART DOCUMENTS

### Patent Literatures

[Patent Literature 1] WO2008/038579
[Patent Literature 2] WO2011/096482
[Patent Literature 3] WO2011/021503

### Non Patent Literatures

[Non-Patent Literature 1] Watarai et al., Blood 115:230-237, 2010.
[Non-Patent Literature 2] Vizcardo et al., Cell Stem Cell. 12: 31-36. 2013.
[Non-Patent Literature 3] Nishimura T et al., Cell Stem Cell. 12: 114-226. 2013.
[Non-Patent Literature 4] Morgan R. A. et al, Science, 314:126. 2006
[Non-Patent Literature 5] Okamoto S et al, Cancer Res 69:9003, 2009
[Non-Patent Literature 6] Chapuis et al, Sci Transl Med, 5:174ra27, 2013
[Non-Patent Literature 7] Blood, 116: 5045, 2010
[Non-Patent Literature 8] Ito et al Lancet, 331: 413, 1988
[Non-Patent Literature 9] CYRANOSKI, Nature vol. 488, 139(2012)
[Non-Patent Literature 10] Takahashi and Yamanaka, Cell 126, 663-673 (2006)
[Non-Patent Literature 11] Takahashi et al., Cell 131, 861-872 (2007)
[Non-Patent Literature 12] Grskovic et al., Nat. Rev. Drug Dscov. 10, 915-929 (2011)
[Non-Patent Literature 13] Morgan R. A. et al, Science, 314:126. 2006
[Non-Patent Literature 14] Timmermans et al., Journal of Immunology, 2009, 182: 6879-6888
[Non-Patent Literature 15] Blood 111:1318(2008)
[Non-Patent Literature 16] Nature Immunology 11: 585 (2010)

The prior art documents listed above are herein incorporated by reference.

### SUMMARY OF INVENTION

An object of the present application is to provide a cell-based immunotherapy that is more efficient and safe than conventional immunotherapies.

In one embodiment, a cell-based immunotherapy method which comprises, inducing T cell progenitors or mature T cells from pluripotent stem cells bearing genes encoding a T cell receptor specific for a desired antigen, and allogenically transplanting the T cell progenitors or mature T cells to a patient in need thereof.

In the cell-based immunotherapy according to the present application, the T cells having the desired antigen specificity may be prepared by inducing iPS cells from a T cell having the desired antigen specificity, differentiating the iPS cells into T cell progenitors or mature T cells, and then, the obtained cells are subjected to the allograft. In this specification, iPS cells induced from a T cell is called as “T-iPS cells”.

In general, the antigen specific T cells are expected to be isolated from the patient suffered from an infectious disease or a cancer. This is because the antigen specific T cells are amplified in the body of the patient and therefore, it could be easy to detect and obtain the T cells with a specific reactivity. According to the present application, a method for preparing T-iPS for allograft, which comprises: obtaining a T cell specific for a disease-relating antigen from a patient who is suffered from the disease and, preparing T-iPS cells to be used for the allograft from the T cell. The present application further provides a method which comprises the step of obtaining an antigen specific T cell from a healthy volunteer. By employing T-iPS cells from a T cell of the healthy volunteer, various advantages can be obtained:

1) T cells with various antigen specificities can be induced from the cells of a healthy volunteer and therefore, T-iPS cells bearing various kinds of TCR genes can be previously prepared.

2) It will be easier to collect donors from healthy volunteers for creating a T-iPS bank.

The T cells used in the cell-based immunotherapy are clonally expanded T cell population and therefore, all of the cells in the population bear the single TCR. Accordingly, the possibility of causing a graft-versus-host reaction is significantly low and the cells can be used not only for autologous transplantation but also for allogenic transplantation. The art could not expect the method provided herein in view of the commonsense that “allogenic transplantation of T cells is an absolute contraindication”.

According to the cell-based immunotherapy method of this application, T cell progenitors or mature T cells are transplanted to a patient having HLAs that match the HLAs of the donor to a predetermined extent. In the cell-based immunotherapy method of the present application, the lymphocytes derived from the patient to be treated and regenerated T cells to be transplanted may be co-cultured before the transplantation to confirm whether or not the regenerated T cells have allogenic reactivity against the patient. As discussed above, the T cells used for the cell-based immunotherapy will be provided as a clonally expanded cell population and therefore, the risk of exerting graft-versus-host-reaction against the patient's body is low. However, the risk that the regenerated T cells trigger an allogenic reaction against the patient is not zero. For the safety reason, the regenerated T cells and the lymphocytes obtained from the patient to be treated may be co-cultured to confirm that regenerated T cells do not exert allogenic reactivity against the patient's HLA.

Further, the present application provides a method for inducing T cells for a cell-based immunotherapy, which comprises the steps of:

(1) providing human pluripotent stem cells bearing a T cell receptor specific for a desired antigen, and

(2) inducing T cell progenitors or mature T cells from the pluripotent stem cells of step (1).

According to another embodiment, a method for cell-based immunotherapy, further comprises the step of: co-culturing the T cells induced from the pluripotent stem cells with lymphocytes derived from the subject to be treated by the cell-based immunotherapy to confirm the allogenic reactivity of the T cells against the subject is provided.

According to the present application, human pluripotent stem cells may preferably be human iPS cells.

### Effect of the Invention

According to the present application, the inventors could unexpectedly solve the above recognized problems to some extent. The following effects are available:

1) No need for preparing T cells for transplantation for each patient. Therefore, preparation for the cell-based immunotherapy can be conducted previously.

2) The treatment can be started after the safety and quality of the cells to be transplanted are verified.

3) Even if an allograft between the HLA-match patient and donor, some minor antigens do not match and therefore, the transplanted cells are eventually rejected by the patient's immune reaction. A safe treatment with significantly less risk of canceration of the transplanted cells can be conducted.

### BRIEF DESCRIPTION OF DRAWINGS

FIG. 1 is a result of FACS analysis of the cells obtained in Example 1. LMP2 tetramer positive-CD8 positive T cells were induced from T cells of a healthy volunteer.

FIG. 2 shows that T cells induced by using LMP2 peptide from peripheral blood obtained from a healthy volunteer having HLA-A2402 who had previously been infected with EB virus exerted the peptide specific killer activity in example 1.

FIG. 3 is a photograph of an iPS cell colony induced from a LMP2 peptide specific T cell.

FIG. 4 is a result of FACS analysis of the cells on day 13 of the differentiation of T-iPS cells established from LMP2 peptide specific T cells into T cells.

FIG. 5 is a result of FACS analysis of the cells on day 36 of the differentiation of T-iPS cells established from LMP2 peptide specific T cells into T cells.

FIG. 6 is a result of FACS analysis of the cells on day 41 of the differentiation of T-iPS cells established from LMP2 peptide specific T cells into T cells. Generation of LMP2 specific mature T cells (CTLs) was confirmed.

FIG. 7 shows LMP2 specific killer activity of the mature T cells (CTLs) re-generated from T-iPS cells established from a LMP2 peptide specific T cell. The killer activities in the presence (p+) or absence (p−) of LMP2 peptide were observed by using LCLs as target cells.

FIG. 8 shows natural killer cell-like activities of mature T cells re-generated from T-iPS cells established from a LMP2 peptide specific T cell.

FIG. 9 shows peptide specific cytotoxicity of CTLs regenerated from clone LMP2#1 against LCLs.

FIG. 10 shows peptide specific cytotoxicity of CTLs regenerated from clone LMP2#13 obtained in Example 2 against LCLs.

FIG. 11 shows that WT1 tetramer positive-CD8 positive T cells population were induced from T cells derived from a healthy volunteer in example 3. Result of FACS analysis.

FIG. 12 is a photograph of an iPS cell colony established from a WT1 peptide specific T cell.

FIG. 13 is a result of FACS analysis of the cells on day 13 of the differentiation of T-iPS cells established from WT1 peptide specific T cells into T cells.

FIG. 14 is a result of FACS analysis of the cells on day 36 of the differentiation of T-iPS cells established from WT1 peptide specific T cells into T cells.

FIG. 15 shows peptide specific cytotoxicity of the CTLs regenerated from clone WT1#9 obtained in Example 3 against LCLs.

FIG. 16 shows peptide specific cytotoxicity of the CTLs regenerated from clone WT1#3-3 obtained in Example 4 against LCLs.

FIG. 17 shows cytotoxic activity of the CTLs regenerated from clone WT1#3-3 against THP1 leukemia cells. The cytotoxic activity of the cells was completely blocked by an anti-HLA class I antibody.

FIG. 18 shows cytotoxic activity of the CTLs regenerated from clone WT1#3-3 against HL60 leukemia cells. The cytotoxic activity of the cells was completely blocked by an anti-HLA class I antibody.

FIG. 19 shows the result of the non-growth control in Example 5. The regenerated CTLs were cultured in the presence of IL-7 (5 ng/mL) only.

FIG. 20 shows the result obtained without the target cells (control) in Example 5. The cells a little proliferated even without the target cells. In this example, the proliferated amount was used as control.

FIG. 21 supports that the regenerated CTLs did not exert allogenic reaction against the autologous HLA.

FIG. 22 supports that the regenerated CTLs in general do not exert allogenic reaction against HLAs of a third person.

FIG. 23 supports that the regenerated CTLs may exert allogenic reaction against HLAs of a third party.

### DETAILED DESCRIPTION OF PREFERRED EMBODIMENTS

In the specification and claims, “pluripotent stem cells” refer to stem cells having pluripotency, i.e. an ability to differentiate into many types of cells in the body, and self-propagation ability. Examples of pluripotent stem cells may include embryonic stem cells (ES cells), nuclear transfer embryonic stem cells (ntES cells), embryonic germ cells (EG cells), and induced pluripotent stem cells (iPS cells). iPS cells and Muse cells are preferable in view of the fact that those pluripotent stem cells can be obtained by not destroying the embryos The pluripotent stem cells are preferably those derived from mammal and more preferably, are human pluripotent stem cells. According to the present application, pluripotent stem cells are preferably those derived from a mammal and especially from a human. iPS cells are preferably used. In the specification and claims, iPS cells induced from a T cell is called as “T-iPS cells”

In the specification and claims, “T cells” refer to cells expressing a receptor for an antigen called as T cell receptor (TCR). The fact that TCR of a T cell is maintained in iPS cells induced from the T cell has been reported by WO2011/096482 and Vizcardo et al., Cell Stem Cell 12, 31-36 2013.

T cells used as origin for iPS cells nay preferably be T cells expressing at least one of CD4 and CD8, in addition to CD3. Examples of the preferable human T cells my include helper/regulatory T cells that are CD4 positive cells; cytotoxic T cells that are CD8 positive cells; naive T cells that are CD45RA⁺CD62L⁺ cells; central memory T cells that are CD45RA⁻CD62L⁺ cells, effector memory T cells that are CD45RA⁻CD62T⁻ cells and terminal effector T cells that are CD45RA⁺CD62L⁺ cells.

Human T cells can be isolated from a human tissue by known procedures. The human tissue is not limited in particular, if the tissue contains T cells of the above-mentioned type, and examples thereof include peripheral blood, lymph node, bone marrow, thymus, spleen, umbilical cord blood, and a lesion site tissue. Among these, peripheral blood and umbilical cord blood are preferable since they can be derived less invasively from the human body and can be prepared with ease. Known procedures for isolating human T cells include, for example, flow cytometry using an antibody directing to a cell surface marker, such as CD4, and a cell sorter, as shown in the below-mentioned Examples. Alternatively, desired T cells can be isolated by detecting the secretion of a cytokine or the expression of a functional molecule as an indicator. In this case, for example, T cells secrete different cytokines, depending on whether they are of the Th1 or Th2 type, and thus T cells of a desired Th type can be isolated by selecting T cells using the cytokine as an indicator. Similarly, cytotoxic (killer) T cells can be isolated using the secretion or production of granzyme, perforin, or the like as an indicator.

“T cell specific for a desired antigen” and “T cell bearing a TCR specific for a desired antigen” may be obtained from a donor by deriving or inducing cytotoxic T lymphocytes bearing the TCR from donor cells. For example, cytotoxic T lymphocytes specific for a cancer antigen may be obtained by stimulating the lymphocytes conventionally obtained from the donor with the cancer antigen specific for the cancer to be treated. C Cancer antigens have been identified for variety of cancers and procedures for inducing cytotoxic T lymphocytes with a cancer antigen or an epitope peptide thereof have been well known. Alternatively, the lymphocytes may be co-cultured with cells of the cancer to be treated.

Alternatively, cytotoxic T lymphocytes specific for a cancer antigen of a cancer to be treated may be induced from peripheral blood of a subject who is suffered from the cancer.

“Human T cells specific for a desired antigen” may be isolated from human cell culture or human tissue containing T cells specific for the antigen by using an affinity column to which the desired antigen is immobilized. Alternatively, Human T cells specific for a desired antigen may be purified from human tissues by using a tetramer of the antigen-bound major histocompatibility complex (MHC tetramer).

Pluripotent stem cells are induced from a human T cell specific for a desired antigen. The procedure for inducing pluripotent stem cells from a T cell may be those taught by Vizcardo et al., Cell Stem Cell 12, 31-36 2013. For example, T cells specific for the desired antigen may be obtained from an individual who had acquired immunity against the disease to be treated and the Yamanaka factors may be introduced into the T cells to give iPS cells (Takahashi and Yamanaka, Cell 126, 663-673 (2006), Takahashi et al., Cell 131, 861-972(2007) and Grskovic et al., Nat. Rev. Drug Dscov. 10, 915-929 (2011).

Induced pluripotent stem (iPS) cells can be prepared by introducing specific reprogramming factors to somatic cells. iPS cells are somatic cell-derived artificial stem cells having properties almost equivalent to those of ES cells (K. Takahashi and S. Yamanaka (2006) Cell, 126:663-676; K. Takahashi et al. (2007), Cell, 131:861-872; J. Yu et al. (2007), Science, 318:1917-1920; Nakagawa, M. et al., Nat. Biotechnol. 26:101-106(2008); and WO 2007/069666). The reprogramming factors may be constituted by genes or gene products thereof, or non-coding RNAs, which are expressed specifically in ES cells; or genes or gene products thereof, non-coding RNAs or low molecular weight compounds, which play important roles in maintenance of the undifferentiated state of ES cells. Examples of genes included in the reprogramming factors include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tell, beta-catenin, Lin28b, Sal11, Sal14, Esrrb, Nrba2, Tbx3 and Glis1, and these reprogramming factors may be used either individually or in combination. Examples of the combination of the reprogramming factors include those described in WO2007/069666; WO2008/118820; WO2009/007852; WO2009/032194; WO2009/058413; WO2009/057831; WO2009/075119; WO2009/079007; WO2009/091659; WO2009/101084; WO2009/101407; WO2009/102983; WO2009/114949; WO2009/117439; WO2009/126250; WO2009/126251; WO2009/126655; WO2009/157593; WO2010/009015; WO2010/033906; WO2010/033920; WO2010/042800; WO2010/050626; WO2010/056831; WO2010/068955; WO2010/098419; WO2010/102267; WO2010/111409; WO2010/111422; WO2010/115050; WO2010/124290; WO2010/147395; WO2010/147612; Huangfu D, et al. (2008), Nat. Biotechnol., 26: 795-797; Shi Y, et al. (2008), Cell. Stem Cell, 2: 525-528; Eminli S, et al. (2008), Stem Cells. 26:2467-2474; Huangfu D, et al. (2008), Nat Riotechnol. 26: 1269-1275; Shi Y, et al. (2008), Cell Stem Cell, 3, 568-574; Zhao Y, et al. (2008), Cell Stem Cell, 3:475-479; Marson A, (2008), Cell Stem Cell, 3, 132-135; Feng B, et al. (2009), Nat Cell Biol. 11:197-203; R. L. Judson et al. (2009), Nat. Biotech., 27:459-461; Lyssiotis C A, et al. (2009), Proc Natl Acad Sci USA. 106:8912-8917; Kim J3, et al. (2009), Nature. 461:649-643; Ichida J K, et al. (2009), Cell Stem Cell. 5:491-503; Heng J C, et al. (2010), Cell Stem Cell. 6: 167-74; Han J, et al. (2010), Nature. 463:1096-100; Mali P, et al. (2010), Stem Cells. 28:713-720, and Maekawa M, et al. (2011), Nature. 474:225-9. The contents of the documents cited in this paragraph are herein incorporated by reference.

The reprogramming factors may be contacted with or introduced into the somatic cells by a known procedure suitable for the form of the factor to be used.

In the case where the reprogramming factors are in the form of protein, the reprogramming factors may be introduced into somatic cells by a method such as lipofection, fusion with a cell-permeable peptide (e.g., HIV-derived TAT or polyarginine), or microinjection.

In the case where the reprogramming factors are in the form of DNA, the reprogramming factors may be introduced into somatic cells by a method such as use of a vector including virus, plasmid and artificial chromosome vectors; lipofection; use of liposome; or microinjection. Examples of the virus vector include retrovirus vectors, lentivirus vectors (these are described in Cell, 126, pp. 663-676, 2006; Cell, 131, pp. 861-872, 2007; and Science, 318, pp. 1917-1920, 2007), adenovirus vectors (Science, 322, 945-949, 2008), adeno-associated virus vectors and Sendai virus vectors (WO2010/008054). Examples of the artificial chromosome vector include human artificial chromosome (HAC), yeast artificial chromosome (YAC), and bacterial artificial chromosome (BAC and PAC). Examples of the plasmid which may be used include plasmids for mammalian cells (Science, 322:949-953, 2008). The vector may contain a regulatory sequence(s) such as a promoter, enhancer, ribosome binding sequence, terminator and/or polyadenylation site to enable expression of the nuclear reprogramming factors; and, as required, a sequence of a selection marker such as a drug resistance gene (e.g., kanamycin-resistant gene, ampicillin-resistant gene or puromycin-resistant gene), thymidine kinase gene or diphtheria toxin gene; a gene sequence of a reporter such as the green-fluorescent protein (GFP), β-glucuronidase (GUS) or FLAG. Further, in order to remove, after introduction of the gene into the somatic cells and expression of the same, the genes encoding the reprogramming factors, or both the promoter(s) and the genes encoding the reprogramming factors linked thereto, the vector may have LoxP sequences upstream and downstream of these sequences. The documents cited in this paragraph are herein incorporated by reference.

Further, in the case where the reprogramming factors are in the form of RNA, each reprogramming factor may be introduced into somatic cells by a method such as lipofection or microinjection, and an RNA into which 5-methylcytidine and pseudouridine (TriLink Biotechnologies) were incorporated may be used in order to suppress degradation (Warren L, (2010) Cell Stem Cell. 7:616-630). The documents cited in this paragraph are herein incorporated by reference.

Examples of the medium for inducing iPS cells include DMEM, DMEM/F12 and DME media supplemented with 10 to 15% FBS (these media may further contain LIF, penicillin/streptomycin, puromycin, L-glutamine, non-essential amino acids, β-mercaptoethanol and/or the like, as appropriate); and commercially available media. Examples of the commercially available media include medium for culturing mouse ES cells (TX-WES medium, Thromb-X), medium for culturing primate ES cells (medium for primate ES/iPS cells, ReproCELL) and serum-free medium (mTeSR, Stemcell Technology)].

Examples of the method to induce iPS cells include a method wherein somatic cells and reprogramming factors are brought into contact with each other at 37° C. in the presence of 5% CO₂ on DMEM or DMEM/F12 medium supplemented with 10% FBS, and the cells are cultured for about 4 to 7 days, followed by plating the cells on feeder cells (e.g., mitomycin C-treated STO cells or SNL cells) and starting culture in a bFGF-containing medium for culturing primate ES cells about 10 days after the contact between the somatic cells and the reprogramming factors, thereby allowing ES-like colonies to appear about 30 to about 45 days after the contact, or later.

Alternatively, the cells may be contacted with the reprogramming factors and cultured at 37° C. in the presence of 5% C0₂ on feeder cells (e.g., mitomycin C-treated STO cells or SNL cells) in DMEM medium supplemented with 10% FBS (this medium may further contain LIF, penicillin/streptomycin, puromycin, L-glutamine, non-essential amino acids, R-mercaptoethanol and the like, as appropriate) for about 25 to about 30 days or longer, thereby allowing ES-like colonies to appear. Preferred examples of the culture method include a method wherein the somatic cells themselves to be reprogrammed are used instead of the feeder cells (Takahashi K, et al. (2009), PLoS One. 4:e8067 or WO2010/137746), and a method wherein an extracellular matrix (e.g., Laminin-5 (WO2009/123349), Laminin-5 (WO2009/123349), Laminin-10 (US2008/0213885) or its fragment (WO2011/043405) or Matrigel (BD)) is used instead. The documents cited in this paragraph are herein incorporated by reference.

Other examples include a method wherein the iPS cells are established using a serum-free medium (Sun N, et al. (2009), Proc Natl Acad Sci USA. 106: 15720-15725). Further, in order to enhance the establishment efficiency, iPS cells may be established under low oxygen conditions (at an oxygen concentration of 0.1% to 15%) (Yoshida Y, et al. (2009), Cell Stem Cell. 5:237-241 or WO2010/013845). The contents of the documents cited in this paragraph are herein incorporated by reference.

Examples of factors used for enhancing the establishment efficiency may include histone deacetylase (HDAC) inhibitors [e.g., low-molecular inhibitors such as valproic acid (VPA), trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene) and the like), and the like], MEK inhibitor (e.g., PD184352, PD98059, U0126, SL327 and PD0325901), Glycogen synthase kinase-3 inhibitor (e.g., Bio and CHIR99021), DNA methyl transferase inhibitors (e.g., 5-azacytidine), histone methyl transferase inhibitors [for example, low-molecular inhibitors such as BIX-01294, and nucleic acid-based expression inhibitors such as siRNAs and shRNAs against Suv39h1, Suv39h2, SetDB1 and G9ai, L-channel calcium agonist (for example, Bayk8644), butyric acid, TGF3 inhibitor or ALK5 inhibitor (e.g., LY364947, SB431542, 616453 and A-83-01), p53 inhibitor (for example, siRNA and shRNA against p5³), ARID3A inhibitor (e.g., siRNA and shRNA against ARID3A), miRNA such as miR-291-3p, miR-294, miR-295, mir-302 and the like, Wnt Signaling (for example, soluble Wnt3a), neuropeptide Y, prostaglandins (e.g., prostaglandin E2 and prostaglandin J2), hTERT, SV40LT, UTF1, IRX6, GLIS1, PITX2, DMRTB1 and the like. Upon establishing iPS cells, a medium added with the factor for enhancing the establishment efficiency may be used.

During the culture, the medium is replaced with the fresh medium once every day from Day 2 of the culture. The number of somatic cells used for nuclear reprogramming is not restricted, and usually within the range of about 510; to about 5×10⁶ cells per 100 cm area on the culture plate.

iPS cells may be selected based on the shape of each formed colony. In the cases where a drug resistance gene is introduced as a marker gene such that the drug resistance gene is expressed in conjunction with a gene that is expressed when a somatic cell was reprogrammed (e.g., Oct3/4 or Nanog), the established iPS cells can be selected by culturing the cells in a medium containing the corresponding drug (selection medium). Further, iPS cells can be selected by observation under a fluorescence microscope in the cases where the marker gene is the gene of a fluorescent protein. Thus induced iPS cells (T-iPS cells) bear the T cell receptor genes derived from the original T cell from which the iPS cells were induced.

Then, the iPS cells bearing genes encoding the desired antigen specific TCR are differentiated into T cell progenitors or mature T cells. The procedure for differentiating pluripotent stem cells into T cell progenitors or mature T cells may be that taught by Timmermans et al., Journal of Immunology, 2009, 182: 6879-6883.

In the specification and claims, “T cell progenitors” may cover cells at any stages of the T cell development, from undifferentiated cells corresponding to hematopoietic stem cells to the cells at the stage just before the cells undergo positive selection/negative selection. Details of the differentiation of T cells are explained in Blood 111:1318(2008) and Nature Immunology 11: 585(2010).

T cells are roughly divided into αβ T cells and γδ T cells. αβ T cells include killer T cells and helper T cells. In this specification and claims “T cells differentiated from iPS cells” cover all types of T cells including T progenitor cells and mature T cells. Preferably, T cells may be those expressing at least one of CD4 and CD8 in addition to CD3.

T cell progenitors or mature T cells differentiated from iPS cells bearing genes encoding the desired antigen specific TCR may be obtained as clonally expanded cells having the same antigen specificity as the original T cell from which the iPS cells were induced. The T cell population to be transplanted will have single antigen specificity. The risk that the T cells cause a graft-versus-host reaction when allogenically transplanted is low and therefore, the cell-based immunotherapy can be conducted safely with the regenerated T cells.

In the method of the present application, the re-generated T cell progenitors or mature T cells are dispersed in a suitable medium such as saline or PBS and the dispersion may be administered to a patient. The matching level of the donor and the patient may be complete match. When the donor is homozygous for HLA haplotype (hereinafter referred to as “HLA haplotype homo”) and the patient is heterozygous for HLA haplotypes (hereinafter referred to as “HLA haplotype hetero”), one of the patient's HLA haplotypes should match the donor's homozygous HLA haplotype.

According to the method provided herein, the induced T cell progenitors or mature T cells are preferably verified that the cells will not cause graft-versus-host reaction in a patient before the T cells are transplanted into the patient. In order to verify the safety of the induced T cell progenitors or mature T cells, Mixed Lymphocyte Reaction (MLR) may be conducted before the transplantation. In detail, the cells may be mixed and co-cultured with cells derived from a tissue of the patient to be transplanted with the T cells, preferably, with lymphocytes of the patient. When regenerated T cell progenitors or mature T cells differentiated from T-iPS cells recognize an HLA of the patient's lymphocytes as an allogenic antigen, the regenerated T cells are activated and proliferate. In such a case, the regenerated T cells are not safe for the cell-based immunotherapy in the patient. On the other hand, the regenerated T cells do not recognize HLAs of the patient's lymphocytes as allogenic antigens, the regenerated T cells will not cause graft-versus-host reaction and can safely be administered to the patient.

The cells may be administered intravenously. The number of the cells to be administered is not limited and may be determined based on, for example, the age, sex, height and body weight of the patient and disease and conditions to be treated. The optimal cell number may be determined through clinical studies.

T cells may target various antigens and therefore, the method of this application may be applied for a cell-based immunotherapy against various diseases including cancers, infectious diseases, autoimmune diseases and allergies. For example, a high proportion of hematopoietic organ tumors such as leukemia, myelodysplastic syndrome, multiple myeloma, and malignant lymphoma, as well as solid tumors such as stomach cancer, colon cancer, lung cancer, breast cancer, germ cell cancer, liver cancer, skin cancer, bladder cancer, prostate cancer, uterine cancer, cervical cancer and ovarian cancer express the WT1 gene. Accordingly, CTLs regenerated from T-iPS cells that are induced from a CTL with WT1 specific cytotoxicity are effective for the cell-based immunotherapy on various WT1 gene expressing cancers.

Epstein-Barr (EB) virus causes various diseases such as infectious mononucleosis as well as cancers such as malignant lymphoma or burkitt lymphoma and epipharyngeal carcinoma. CTLs regenerated from T-iPS cells that are induced from a CTL with cytotoxicity specific for a LMP2 antigen that is an EB virus associated antigen may be useful for the cell-based immunotherapy on various EB virus associated infectious diseases or cancers.

In various proposed therapies wherein various cells or tissues, other than T cells, that are differentiated from iPS cells are transplanted, the cells to be transplanted cells are expected to be fixed in the body of the patient for his/her entire life. In regenerative therapies that use cells or tissues regenerated from iPS cell stock for allogenic transplantation, the patients need to take immune suppressing drugs for their entire life. This is disadvantageous point compared to autologous transplantation. On the other hand, according to the present application, the allogenically transplanted T cells are eventually rejected after a certain period. That is, allogenic graft will be eventually rejected based on mismatches of minor histocompatibility antigens even in the HLA-matched donor and recipient. In this point, the cell-based immunotherapy provided by this application is advantageous than the other proposed allogenic transplantation of the cells or tissues regenerated from iPS cells.

Further, the present method does not require the preparation of the cells for each patient. Previously prepared T-iPS cells having the desired antigen specificity, or T cell progenitors or mature T cells regenerated from the T-iPS cells may be stocked and used. Accordingly, this method has advantages not only of shortening the period for preparation of the cell-based immunotherapy but also enabling the verification of the quality of the cells before transplantation.

For example, T cells specific for a cancer antigen for the treatment of the cancer may be prepared. Specifically, T-iPS cells specific for a cancer antigen may be established from a patient suffered from the cancer. The effect of the T cells regenerated from the T-iPS cells may be previously verified by transferring the T cells regenerated from the T-iPS cells into the patient. Then, the verified T-iPS cells may be stored to create a cell bank. The T-iPS cells stored in the bank can be used for the treatment of a HLA-matched patient suffered from a cancer expressing the same cancer antigen. T cells regenerated from the T-iPS cells can be administered to the patient. If the re-generated T cells are frozen and stored, the time period required for starting the therapy can be shortened and

In this specification, examples in which iPS cells were establishing from a T cell to give “T-iPS cells” are provided. TCR-induced iPS cells obtained by inducing genes encoding a TCR specific for a desired antigen may also be used in the same manner.

### Example 1

T-iPS cells (clone LMP2#1) were established from a T cell specific for LMP2 antigen derived from peripheral blood mononuclear cells of an EB virus carrier. T-iPS cells were differentiated into LMP2 antigen specific CTLs (herein after, referred to as “re-generated LMP2-CTL#1”).

EB virus infection in acute phase may cause infectious mononucleosis and sometimes cause cancer such as barkit: lymphoma. In this example, the donor for T cells was a healthy person who had previously been infected with EB virus. Once infected, this virus stays in the lymphocytes for entire life and therefore, the donor is an EB virus carrier. The donor is, therefore, considered to have chronic EB virus infection.

1) Propagation of cytotoxic T Lymphocytes (CTL) specific for LMP2 antigen
i) The following media were used.

Medium for dendritic cells: CellGro (CellGenix)

TABLE 1
Medium for T cells (T cell medium):

| | Amount | Final conc. |
|---|---|---|
| RPMI | 45 ml | |
| human AB serum | 5 ml | 10% |
| Total | 50 ml | |

ii) The LMP2 antigen peptide used is as follows.

LMP2: IYVLVMLVL (SEQ ID NO: 1)

LMP2 tetramer was purchased from MBL.

iii) The LCL (Lymphoblastoid cell line) used is as follows.

Lymphoblastoid cell line (LCL) established from healthy volunteer A who had previously infected with EB virus and had HLA-A*02:06/24:02; B*39:01/40:02; C*07:02/15:02; DRB1*04:10/09:01 in the Department of Hematology and Oncology, Graduate School of Medicine, Kyoto University, Kyoto, Japan was used as antigen presenting cells.

A. Induction of Human Monocyte Dendritic Cells (MoDC) from Human Peripheral Blood

1. Peripheral blood was obtained from healthy volunteer A having HLA-A2402 who had previously been infected with EB virus. Monocytes were isolated from the blood by using CD14 microbeads. The cells were washed and added with the medium for dendritic cells to give a 5×10⁵ cells/mL suspension.

2. Cytokines were added to the cell suspension to give final concentrations of GM-CSF 800 U/mL (or 50 nq/mL), IL-4 200 U/mL (or 40 ng/mL). Five milliliter (5 mL) of the cell suspension was seeded to each well of a 6-well plate. The plate was incubated at 37° C. with 5% CO₂.

3. The plate was incubated for 3 days and on day 3, 2.5 mL of the culture supernatant was gently removed. Fresh medium for dendritic cells were added with GM-CSF and IL-4 to give final concentrations of 800 U/mL and 200 U/mL respectively.

4. Thus prepared fresh medium for dendritic cells 3 mL was added to each well.

5. On day 6, immature monocyte-derived dendritic cells (MoDCs) were collected from the plate and added in a small amount of fresh medium for dendritic cells.

6. The density of the cell suspension was adjusted to 5×10⁵ cells/mL.

7. GM-CSF (final concentration: 800 U/mL), IL-4 (final concentration: 200 U/mL), TNF-alpha (final concentration: 10 ng/mL), and PGE2 (final concentration: 1 μg/mL) were added to the cell suspension. About 5×10⁵ cells/mL/well of the cell suspension was added to each well of a 24-well plate.

8. The plate was incubated at 37° C. with 5% CO2 for 24 hours.

9. The peptide was added to each well in last 2 hours of the 24 hours incubation period. The final concentration of the peptide was 10 μM. Dendritic cells (DC) were collected from the plate and washed twice with the medium for T cells.

10. The number of the DCs was counted and the medium for T cells was added to give a 2×10⁵ cells/mL suspension.

B. Isolation of T Cells from Human Peripheral Blood and Co-Culture of the T Cells and Dendritic Cells.

1. T cells were isolated from peripheral blood of the healthy volunteer A (the same person in the step A above) by means of the MACS technique using CD3 microbeads. The cells were washed and added with the medium for T cells to give a 2×10⁶ cells/mL suspension. A small part of the T cell suspension was separated for the flow cytometry analysis.

2. 0.5 mL/well of DC cell suspension (2×10⁵ cells/mL) and 0.5 mL/well of the T cell suspension (2×10⁶ cells/mL) were added to each well of a 24 well plate. (DC cells: T cells=1×10⁵:1×10⁶=1:10).

3. On day 3, IL-7 (final concentration: 5 ng/mL) and IL-15 (final concentration: 10 ng/mL) were added to each well.

4. On day 14, the cells were collected from the culture.

### C. Addition of the Peptide to LCL

1. LCLs were collected from the culture and irradiated at a dose of 35Gy.

2. The irradiated cells were suspended in the T cell medium to give a 5×10⁵ cells/mL suspension.

3. The peptide was added to the suspension 100 nM and incubated for 2 hours.

4. The LCL were collected and washed with the T cell medium and then, dispersed in the T cell medium to give a 2×10⁵ cells/mL suspension.

D. Co-Culture of LCL and T Cells Stimulated with the Dendritic Cells.

1. The T cells stimulated with the dendritic cells were dispersed in the T cell medium to give a 2×10⁶ cells/mL suspension.

2. 0.5 mL/well of the LCL suspension (2×10⁵ cells/mL) incubated in the presence of the peptide and 0.5 mL/well of T cell suspension (2×10⁶ cells/mL) were added to each well of a 24-well plate (LCL: T cells=1×10⁵:1×10⁶=1:10). Simultaneously, the peptide was added to the well to give the final concentration of 100 nM.

3. On day 3, IL-7 (final concentration: 5 ng/mL) and IL-15 (final concentration: 1 ng/mL) were added to the well. The plate was incubated for 2 weeks and the medium was changed every week with the fresh T cell medium supplemented with the cytokines. (1st course of stimulation with peptide-pulsed LCL)

4. LCLs were again incubated in the medium supplemented with 100 nM of the peptide for 2 hours and then, added with the CTLs.

5. On day 3, IL-7 (final concentration: 5 ng/mL) and IL-15 (final concentration: 1 ng/mL) were added to the well. The plate was incubated for 2 weeks and the medium was changed every week with the fresh T cell medium supplemented with the cytokines. (2nd course of stimulation with peptide-pulsed LCL)

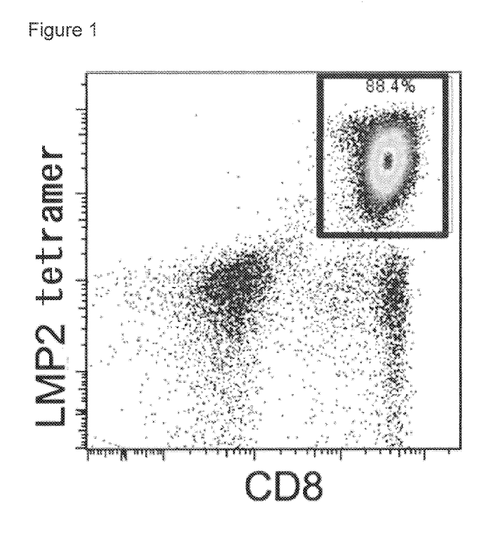

6. Thus obtained cells were analyzed by flow cytometry and confirmed that more than 80% of CD8 positive T cells were CD8 positive and LMP-2 tetramer positive cells. Results are shown in FIG. 1.

### E. Antigen Specific Killer Activity of the LMP2 Specific CTLs

1. CFSE-labelled OUN-1 leukemia cells were used as target cells. The labelled cells were dispersed in the T cell medium and incubated in the presence of 1 nM of the LMP2 peptide for 2 hours.

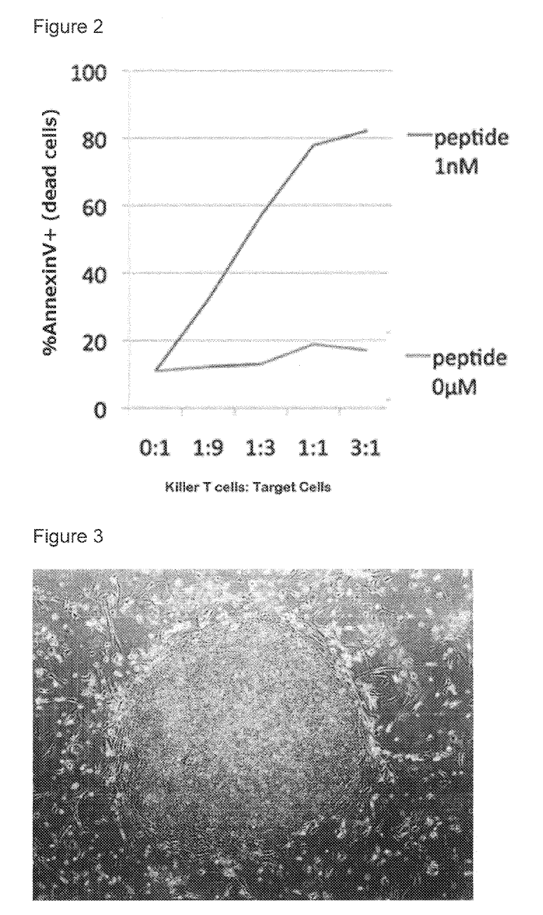

2. LMP2 specific cytotoxic T cells (CD8 positive and LMP-2 tetramer positive cells) expanded under the peptide stimulation and the CFSE-labelled OUN-1 leukemia cells were added to each well of a 96-well round bottom plate at different effector/target cell ratios of 0:1, 1:9, 1:3, 1:1 and 3:1. The cells were incubated in the presence or absence of the peptide. The ratio of Annexin V positive cells to PI (Propidium Iodide) positive cells in the CFSE positive cell fraction were determined to confirm percentage of dead cells among the target cells. Results are shown in FIG. 2.

3. Thus prepared LMP2 specific killer T cells were confirmed to have the antigen specific killer activity against the target cells.

2) Establishment of the LMP2-T-iPS Cells

### A. Activation of LMP2 Specific CTLs.

1. CD8 positive cells were enriched from the above obtained LMP2 specific CTLs by using MACS beads.

2. The enriched cell population was dispersed in the T cell medium and added with IL-7 (final concentration: 5 ng/mL) and IL-15 (final concentration: 10 ng/mL). Dynabeads Human T-Activator CD3/CD28 was added to give a bead-to-cell ratio of 1:1, and the mixture was incubated for 2 days to activate the CD8 positive cells.

### B. Introduction of the Yamanaka Four Factors and SV40 by Means of Sendai Virus Vector.

1. The activated LMP2 specific CTLs were dispersed in the T cell medium, Sendai virus bearing four Yamanaka factors and SV40 was added to the medium and the cell suspension was cultured for 2 days.

2. The obtained cells were washed with the T cell medium and added with the T cell medium supplemented with IL-7 (final concentration: 5 ng/mL) and IL-15 (final concentration: 1 ng/mL). The cells were further cultured for 2 days.

3. After that, all cells were collected and dispersed in the T cell medium supplemented with IL-7 (final concentration: 5 ng/mL) and IL-15 (final concentration: 1 ng/mL). The cell suspension was seeded on the feeder cells.

4. On day 2, a half of the medium was replaced with the fresh iPS cell medium. After that, a half of the medium was replaced with fresh iPS cell medium every day and the cells were continuously cultured.

C. Picking Up iPS Cell Colonies from the Culture

1. Three weeks after the introduction of the Yamanaka factors, colonies of iPS cells were visually observed.

2. Colonies were mechanically picked up with a 200 μl pipette tip.

3. Several clones were established individually and one of them was used as LMP2-T-iPS cells in the example below. Photograph of the colony of an obtained clone is shown in FIG. 3.

3) Induction of T Cells from the LMP2-iPS Cells.

### Media Used are as Follows:

TABLE 2
Medium A: for maintenance of OP9 stromal cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 6.25 | mL | 1% |
| solution* | | | |
| Total | 631.25 | mL | |

*Mixture of Penicillin (10,000 U/ml) and Streptomycin (10,000 μg/ml). The final concentrations were 100 U/ml and 100 μg/ml, respectively.

TABLE 3
Medium B: for inducing differentiation of T cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 5 | mL | 1% |
| solution* | | | |
| hrIL-7 (stock: 10 μg/mL) | 315 | μL | 5 ng/mL |
| hrFlT-3L (stock: 10 μg/mL) | 315 | μL | 5 ng/mL |
| hrSCF (stock: 10 μg/mL) | 630 | μL | 10 ng/mL |
| Total | 631.26 | mL | |

*Mixture of Penicillin (10,000 U/ml) and Streptomycin (10,000 μg/ml). The final concentrations were 100 U/ml and 100 μg/ml, respectively.

TABLE 4
Medium C: for inducing from immature T cells into mature T cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 5 | mL | 1% |
| solution* | | | |
| hrIL-7 (stock: 10 μg/mL) | 315 | μL | 5 ng/mL |
| Total | 630.315 | mL | |

*Mixture of Penicillin (10,000 U/ml) and Streptomycin (10,000 μg/ml). The final concentrations were 100 U/ml and 100 μg/ml, respectively.

### Preparation of OP9 Cells

Six milliliters (6 mL) of 0.1% gelatin solution in PBS was added to a 13 cm dish (Falcon) and incubated for 30 minutes at 37° C. The gelatin solution was then removed and 10 ml of medium A was added to the dish. OP9 stromal cells were obtained from a confluent culture and seeded in the dish. Four days after, medium A 10 mL was added to the dish (final amount was 20 mL).

Induction of Hematopoietic Progenitor Cells from iPS Cells

The medium in the OP9 stromal cell culture to be used for the co-culture was aspirated and replaced with fresh medium A. The medium in the iPS cell culture dish was also aspirated and 10 ml of fresh medium A was added. The iPS cell mass was cut with an EZ-passage roller. The cut iPS cell mass was suspended by using a pipetman with a 200 ul tip. The number of the iPS cell clusters was visually counted and approximately 600 iPS cell clusters were seeded on the OP 9 cells. Three or more dishes per clone of iPS cells were used, and when subculturing, the cells in all dishes were once pooled in one dish and then redistributed to the same number of dishes to reduce the disparity between the dishes.

Day 1: (the Medium was Replaced)

Whether the iPS cell mass adhered to the dish started to differentiate was confirmed. The cell culture medium was replaced with 20 mL of fresh medium A.

Day 5: (a Half of the Medium was Replaced)

A half of the cell culture medium was replaced with 10 mL of fresh medium A.

Day 9: (a Half of the Medium was Replaced)

A half of the cell culture medium was replaced with 10 mL of fresh medium A.

Day 13: (Induced Mesodermal Cells were Transferred from OP9 Cell Layer onto OP9/DLL1 Cell Layer)

Cell culture medium was aspirated to remove and the surface of the cultured cells were washed with HBSS (⁺Mg⁺Ca) to washout the cell culture medium. 10 mL of Collagenase IV 250 U in HBSS (+Mg+Ca) solution was added to the dish and incubated for 45 minutes at 37° C.

The collagenase solution was removed by aspiration and the cells were washed with 10 mL of PBS(−). Then, 0.05% trypsin/EDTA solution was added to the dish and the dish was incubated for 20 minutes at 37° C. After the incubation, the sheet like cell aggregates peeled from the bottom of the dish and the cell aggregates were mechanically fragmented to smaller sizes by means of pipetting. Thus treated cells were added with fresh medium A 20 mL and cultured for more 45 minutes at 37° C.

The culture medium containing the floating cells was passed through 100 μm mesh and the cells were collected. The cells were then centrifuged at 1200 rpm for 7 minutes at 4° C. The obtained pellet was suspended in 10 mL of medium B. One-tenth of the suspension was separated and used for the FACS analysis. The remaining cell suspension was seeded to new dishes containing OP9/DLL1 cells. Cell suspensions obtained from several dishes were pooled and the pooled cells were seeded to the same number of new dishes.

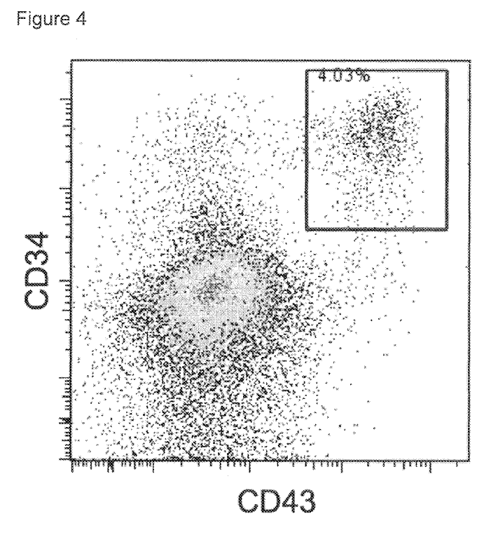

In order to ascertain whether or not hematopoietic progenitor cells were contained in the obtained cells, FACS analysis was carried out using anti-CD34 antibody and anti-CD43 antibody. The results are shown in FIG. 4. Since a sufficient number of cells could be confirmed in the CD34^{low}CD43+ cell fraction, it was confirmed that hematopoietic progenitor cells were induced.

C. Induction of T Cells from Hematopoietic Progenitor Cells.

Then, the obtained cells were seeded on OP9/DLL1 cells. In this step, cell sorting of the CD34^{low}CD43⁺ cell fraction was not performed. When this fraction is sorted, the efficiency of differentiation of T cells could be reduced in comparison with the case where sorting was not performed due to the decrease of the cells or damage to the cells by sorting.

During the culturing period, FACS analysis was conducted several times to confirm the differentiation stages. A considerable number of dead cells were observed over the culturing period. Before the FACS analysis, dead cells were eliminated by using, for example, Propidium Iodide (PI) or 7-AAD.

Day 16: (Cells were Subcultured)

The cells loosely adhered to the OP9 cells were dissociated by gently pipetting several times. The cells were passed through a 100 μm mesh and collected in a 50 mL conical tube. The tube was centrifuged at 1200 rpm for 7 minutes at 4° C. The pellet was dispersed in 10 mL of medium B. Thus prepared cell suspension was seeded on the OP9/DLL1 cells.

Day 23: (Cells were Subcultured) Blood Cell Colonies Began to Appear.

The cells loosely adhered to the OP9/DLL1 cells were dissociated by gently pipetting several times. The cells were passed through a 100 μm mesh and collected in a 50 mL conical tube. The tube was centrifuged at 1200 rpm for 7 minutes at 4° C. The pellet was dispersed in 10 mL of medium B.

Day 36: LMP2 Tetramer Positive Cells were Confirmed

In order to confirm T cells specific for the LMP2 antigen were induced, the cells on Day 36 were analyzed by FACS with anti CD3 antibody and LMP2 tetramer.

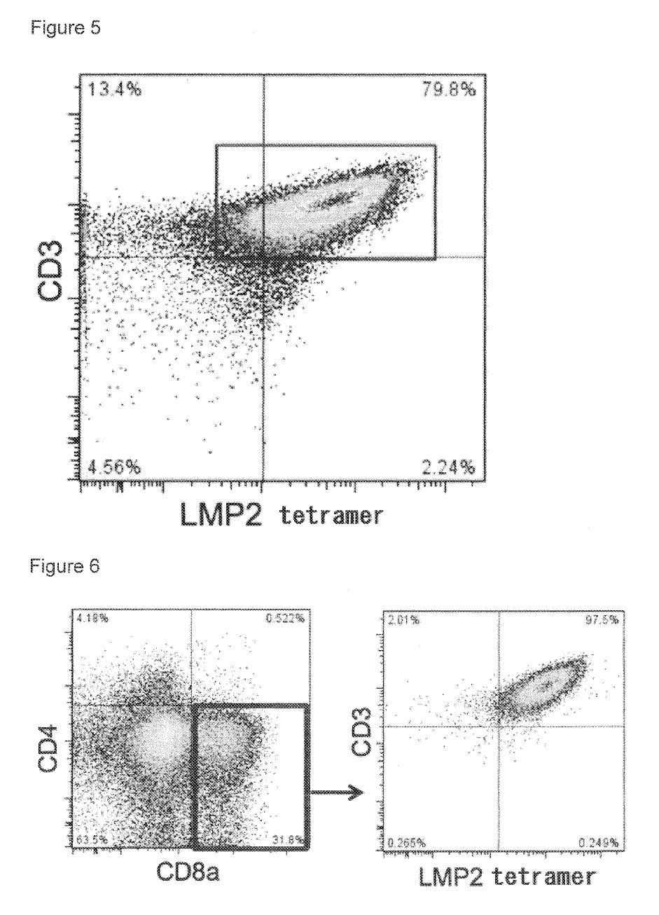

Results are shown In FIG. 5. CD3⁺ cells were observed and a part of the cells were differentiated into CD3 LMP2 tetramer positive cells.

D. Induction of Mature Killer T Cells from the Immature T Cells.

On day 36, LMP2 positive T cells were confirmed with flow cytometry and then, the cells were added with IL-15 so that the cells are differentiated into mature killer T cells or CD8SP cells. The T cells were dispersed in medium C and seeded on the fresh OP9/DLL1 cell layer in each well of a 24-well plate at a density of 3×10⁵ cells/well. IL-15 was added to each well to give final concentration of 10 ng/mL.

Day 41: Mature Killer T Cells were Observed

Five days after the addition of IL-15, the cells were analyzed with FACS. Result is shown in FIG. 6. Mature CD8 single positive cells were observed.

### 4) Antigen-Specific Killer Activity of the Re-Generated LMP2 Specific CTLs

1. CFSE-labelled LCLs were used as target cells. The labelled cells were dispersed in the T cell medium and incubated in the presence of 1 nM of the LMP2 peptide for 2 hours.

2. The regenerated CD8 single positive T cells and the target cells (LCLs) were added to each well of a 96-well round bottom plate at different effector/target cell ratios of 0:1, 1:9, 1:3, 1:1, 3:1, 10:1 and 30:1. The cells were incubated in the presence (p+) or absence (p−) of the peptide. The ratio of Annexin V positive cells to PI (Propidium Iodide) positive cells in the CFSE positive cell fraction were determined to confirm percentage of dead cells among the target cells.

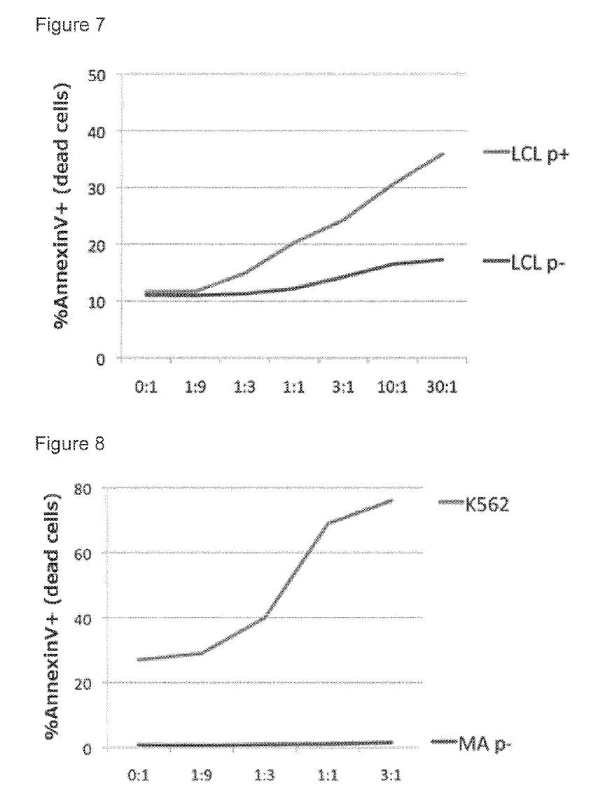

3. Results are shown in FIG. 7. Thus prepared LMP2 specific killer T cells were confirmed to have the antigen specific killer activity against the target cells.

### 5) Natural Killer Cell-Like Activity of the Re-Generated LMP2 Specific CTLs

1. K562 cell line that does not express HLA on the cell surface (to determine alloreactivity) and peripheral blood mononuclear cells of the healthy volunteer A (MA p−) (to determine auto reactivity) were used as target cells. Those cells were labelled with CFSE and suspended in the T cell medium.

2. The regenerated CD8T cells and the target cells were added to each well of a 96-well round bottom plate at different effector/target cell ratios of 0:1, 1:9, 1:3, 1:1, and 3:1. The cells were incubated and the ratio of Annexin V positive cells to PI (Propidium Iodide) positive cells in the CFSE positive cell fraction were determined to confirm percentage of dead cells among the target cells.

3. Results are shown in FIG. 8. The LMP2 specific killer T cells did not kill the autologous PBMC (MA p−) but showed high killer activity against K562 cells. This result support that the LMP2 specific killer T cells have natural-killer cell like activity.

6) Antigen Specific Killer Activity of the Re-Generated LMP2 antigen specific CTLs

1. CFSE labelled LCLs were used as target cells. The cells were suspended in the T cell medium and incubated in the presence of the LMP2 peptide for 2 hours.

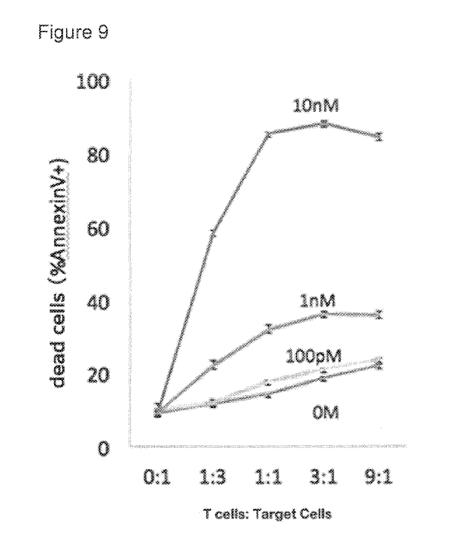

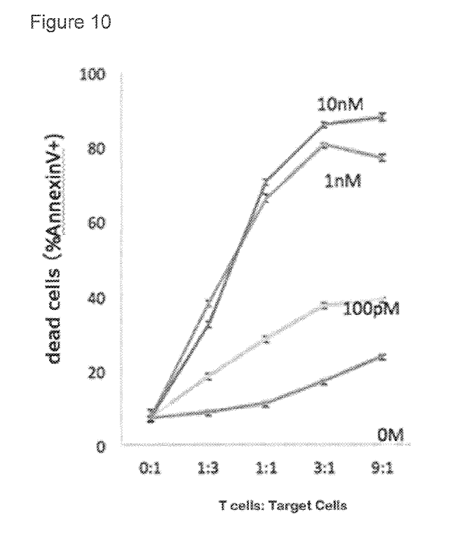

2. The regenerated CD8 single positive T cells (Re-generated LMP2-CTL#1 and the target cells (LCLs) were added to each well of a 96-well round bottom plate at different effector/target cell ratios of 0:1, 1:9, 1:3, 1:1, 3:1 and 9:1. The cells were incubated in the presence of various concentrations of LMP2 peptide or absence of the peptide. After 6 hours incubation, the ratio of Annexin V positive cells to PI (Propidium Iodide) positive cells in the CFSE positive cell fraction were determined to confirm percentage of dead cells among the target cells. Results are shown in FIG. 9.

3. The regenerated LMP2-CTL#1 showed high antigen specific cytotoxic activity against the peptide-loaded LCLs.

### Example 2

LMP2 peptide specific CTLs were induced according to the procedure of Example 1 from a healthy volunteer other than healthy volunteer A from whom PBMC were obtained in Example 1. T-iPS cells (clone LMP2#13) were established from the CTL and then, the T-iPS cells were differentiated into CD8 single positive T cells (re-generated LMP2-CTL#13). The LMP2 peptide used in Example 1 was also used in this example. The antigen specific killer activity of the re-generated CTL cells against the peptide-loaded LCL cells as target cells was determined. Result is shown in Example 10. The healthy volunteer in Example 2 had previously been infected with EB virus and was EBNA antibody positive, and had HLA-A*02:10/24:02; B*07:02/40:06; C*07:02/08:01; DRB1*04:05/04:05.

The regenerated LMP2-CTL (#13) showed high antigen specific cytotoxic activity against the peptide-loaded LCLs.

### Example 3

WT1 antigen specific cytotoxic T cells were induced from peripheral blood of a healthy volunteer, and T-iPS cells (clone WT#9) were established from the CTL. Then, WT1 antigen specific mature T cells (re-generated WT1-CTL(#9)) were induced from the T-iPS cells.

This example comprises the following steps:

1) Amplification of WT1 antigen specific CTLs

2) Establish of WT1-T-iPS cells

3) Induction of CD8 single positive T cells (CTLs) from the WT1-T-iPS cells.

4) Confirmation of antigen specific killer activity of the re-generated WT1-CTL obtained in step 3).

1) Amplification of WT1 antigen specific CTL

i) The following medium was used.

TABLE 5
Medium for T cells (T cell medium):

| | Amount | Final conc. |
|---|---|---|
| RPMI | 45 ml | |
| human AB serum | 5 ml | 10% |
| Total | 50 ml | |

ii) The WT1 antigen peptide used is as follows.

Modified WT1 peptide: CYTWNQMNL (SEQ ID NO: 2) (Cancer Immunol. Immunothera. 51: 614 (2002))

Both WT1 peptide and WT1 tetramer used below were the modified form.

iii) The LCL (Lymphoblastoid cell line) used is as follows.

The LCL having HLA-A2402 which had been established from a healthy volunteer in the Department of Hematology and Oncology, Graduate School of Medicine, Kyoto University, Kyoto, Japan was used.

A. Isolation of T Cells from Human Peripheral Blood and Stimulation of the Cells with the Peptide

1. Peripheral blood was obtained from a healthy volunteer. Monocytes were purified from the blood by using Ficoll and dispersed in the T cell medium. The healthy volunteer has HLA-A*02:01/24:02; 3*15:01/15:11; C*03:03/08:01; DRB1*12:01/12:02.

2. The cell suspension was added to each well of a 96-well round bottom plate in a density of 2.5×10⁵ cells/mL/well, and the peptide was added to give the final concentrations of 10 μm.

3. On day 3, IL-2 (final concentration: 12.5 U/mL), IL-7 (final concentration: 5 ng/mL) and IL-15 (final concentration: 1 ng/mL) were added to the well. The plate was incubated for 2 weeks and the medium was changed every week with the fresh T cell medium supplemented with the cytokines.

### B. Addition of the Peptide to LCLs.

1. LCLs were collected from the culture and irradiated at a dose of 35Gy.

2. The irradiated cells were suspended in the T cell medium to give a 5×10⁵ cells/mL suspension.

3. The peptide 100 nM was added to the suspension and incubated for 2 hours.

4. The LCLs were collected and washed with the T cell medium and then, dispersed in the T cell medium to give a 2×10⁵ cells/mL suspension.

C. Co-Culture of LCL Pulsed with the Peptide and T Cells.

1. The peptide stimulated T cells were collected when they were incubated for two weeks after the peptide stimulation, washed and then dispersed in the T cell medium to give 2×10⁶ cells/mL suspension. A small part of the T cell suspension was separated for the flow cytometer analysis.

2. A LCL suspension (2×10⁵ cells/mL) that had been incubated in the presence of the peptide 0.5 mL/well and the T cell suspension (2×10⁶ cells/mL) 0.5 mL/well were added to each well of a 24 well plate. (LCLs: T cells=1×10⁵: 1×10⁶=1:10).

3. On day 3, IL-2 (final concentration: 12.5 U/mL), IL-7 (final concentration: 5 ng/mL) and IL-15 (final concentration: 1 ng/mL) were added to each well. The plate was incubated for 2 weeks and the medium was changed every week with the fresh T cell medium supplemented with the cytokines. (1st course of stimulation with peptide-pulsed LCL)

4. LCLs were again incubated in the medium supplemented with 100 nM of the peptide for 2 hours and then, added with the CTLs.

5. On day 3, IL-2 (final concentration: 12.5 U/mL), IL-7 (final concentration: 5 ng/mL) and IL-15 (final concentration: 1 ng/mL) were added to each well. The plate was incubated for 2 weeks and the medium was changed every week with the fresh T cell medium supplemented with the cytokines. (2nd course of stimulation with peptide-pulsed LCL)

6. LCLs were again incubated in the medium supplemented with 100 nM of the peptide for 2 hours and then, added with the CTLs.

7. On day 3, IL-2 (final concentration: 12.5 U/mL), IL-7 (final concentration: 5 ng/mL) and IL-15 (final concentration: 1 ng/mL) were added to each well. The plate was incubated for 2 weeks and the medium was changed every week with the fresh T cell medium supplemented with the cytokines. (3rd course of stimulation with peptide-pulsed LCL)

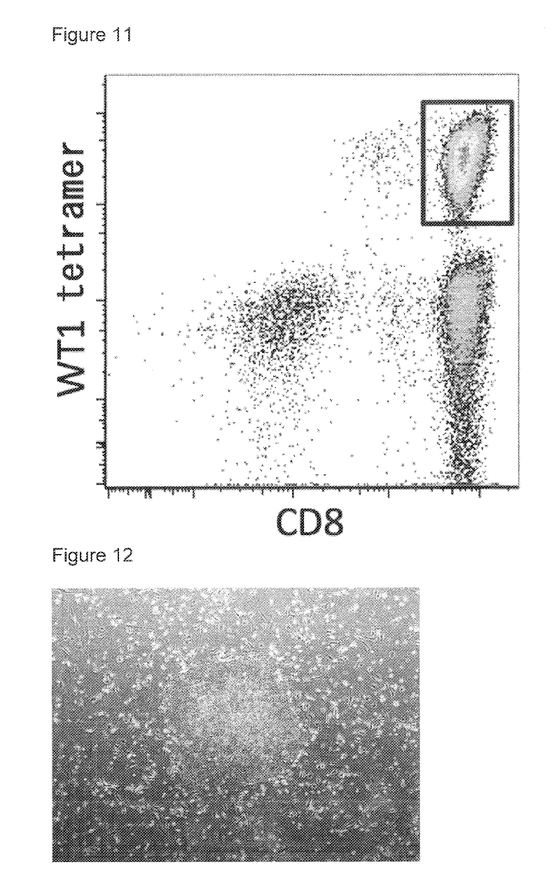

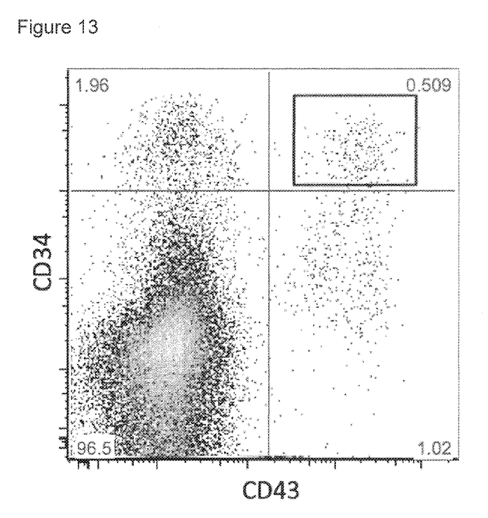

8. Thus obtained cells were analyzed by flow cytometry. The result is shown in FIG. 11. It was confirmed that more than 60% of the CD8 positive T cells were CD8 positive and WT1 tetramer positive cells.

### 2) Establish of the WT1-T-iPS Cells

### A. Activation of WT1 Specific CTLs.

1. CD8 positive cells were enriched from the above obtained WT1 specific CTLs using MACS beads.

2. The enriched cell population was dispersed in the T cell medium and added with IL-2 (final concentration: 12.5 U/mL), IL-7 (final concentration: 5 ng/mL) and IL-15 (final concentration: 1 ng/mL). Dynabeads Human T-Activator CD3/CD28 was added to give a bead-to-cell ratio of 1:1, and the mixture was incubated for 2 days to activate the CD8 positive cells.

### B. Introduction of the Yamanaka Four Factors and SV40 by Means of Sendai Virus Vector.

1. The activated WT1 specific CTLs were dispersed in the T cell medium, Sendai virus bearing four Yamanaka factors and SV40 was added to the medium and the cell suspension was cultured for 2 days.

2. The obtained cells were washed with the T cell medium and added with the T cell medium supplemented with IL-2 (final concentration: 12.5 U/mL), IL-7 (final concentration: 5 ng/mL) and IL-15 (final concentration: 1 ng/mL). The cells were further cultured for 2 days.

3. After that, all cells were collected and dispersed in the T cell medium containing no cytokine. The cell suspension was seeded on the feeder cells.

4. On day 2, a half of the medium was replaced with the fresh iPS cell medium. After that, a half of the medium was replaced with the fresh iPS cell medium every day and the cells were continuously cultured.

C. Picking Up iPS Cell Colonies from the Culture

1. Three weeks after the introduction of the Yamanaka factors, colonies of iPS cells were visually observed.

2. Colonies were mechanically picked up with a 200 μL pipette tip.

3. Several clones were established individually. Photograph of the colony of an obtained clone is shown in FIG. 12.

3) Induction of T Cells from the WT1-T-iPS Cells.
Media used are as follows:

TABLE 6
Medium A: for maintenance of OP9 stromal cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 6.25 | mL | 1% |
| solution* | | | |
| Total | 631.25 | mL | |

*Mixture of Penicillin (10,000 U/ml) and Streptomycin (10,000 μg/ml). The final concentrations were 100 U/ml and 100 μg/ml, respectively.

TABLE 7
Medium B: for inducing differentiation of T cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 5 | mL | 1% |
| solution* | | | |
| hrIL-7 (stock: 10 μg/mL) | 315 | μL | 5 ng/mL |
| hrFlT-3L (stock: 10 μg/mL) | 315 | μL | 5 ng/mL |
| hrSCF (stock: 10 μg/mL) | 630 | μL | 10 ng/mL |
| Total | 631.26 | mL | |

*Mixture of Penicillin (10,000 U/ml) and Streptomycin (10,000 μg/ml). The final concentrations were 100 U/ml and 100 μg/ml, respectively.

### Preparation of OP9 Cells

Six milliliters (6 mL) of 0.1% gelatin solution in PBS was added to a 10 cm dish (Falcon) and incubated for 30 minutes at 37° C. OP9 stromal cells were detached from a confluent culture dish with trypsin/EDTA solution and about ¼ of the obtained cells were added to the gelatin coated 10 cm cell culture dish. 10 mL of medium A was added to the cell culture dish. Four days after, medium A 10 mL was added to the dish (final amount was 20 mL).

Induction of Hematopoietic Progenitor Cells from iPS Cells

The medium in the OP9 stromal cell culture to be used for the co-culture was aspirated and replaced with fresh medium A. The medium in the iPS cell culture dish was also aspirated and 10 ml of fresh medium A was added. The iPS cell mass was cut with an EZ-passage roller. The cut iPS cell mass was suspended by means of a pipetman with a 200 ul tip. The number of the iPS cell clusters was visually counted and approximately 600 iPS cell clusters were seeded on the OP 9 cells. Three or more dishes per clone of iPS cells were used, and when subculturing, the cells in all dishes were once pooled in one dish and then redistributed to the same number of dishes to reduce the disparity between the dishes.

Day 1: (the Medium was Replaced)

Whether the iPS cell mass adhered to the dish started to differentiate were confirmed. The cell culture medium was replaced with 20 mL of fresh medium A.

Day 5: (a Half of the Medium was Replaced)

A half of the cell culture medium was replaced with 10 mL of fresh medium A.

Day 9: (a Half of the Medium was Replaced)

A half of the cell culture medium was replaced with 10 mL of fresh medium A.

Day 13: (Induced Mesodermal Cells were Transferred from OP9 Cell Layer onto OP9/DLL1 Cell Layer)

Cell culture medium was aspirated to remove and the surface of the cultured cells were washed with HBSS(⁺Mg⁺Ca) to washout the cell culture medium. 10 mL of Collagenase IV 250 U in HBSS (+Mg+Ca) solution was added to the dish and incubated for 45 minutes at 37° C.

The collagenase solution was removed by aspiration and the cells were washed with 10 mL of PBS(−). Then, 0.05% trypsin/EDTA solution was added to the dish and the dish was incubated for 20 minutes at 37° C. After the incubation, the sheet like cell aggregates peeled from the bottom of the dish and the cell aggregates were mechanically fragmented to smaller sizes by means of pipetting. Thus treated cells were added with fresh medium A 20 mL and cultured for more 45 minutes at 37° C. The culture medium containing the floating cells was passed through 100 μm mesh and the cells were collected. The cells were then centrifuged at 1200 rpm for 7 minutes at 4° C. The obtained pellet was suspended in 10 mL of medium B. One-tenth of the suspension was separated and used for the FACS analysis. The remaining cell suspension was seeded to new dishes containing OP9/DLL1 cells. Cell suspensions obtained from several dishes were pooled and the pooled cells were seeded to the same number of new dishes.

In order to ascertain whether or not hematopoietic progenitor cells were contained in the obtained cells, FACS analysis was carried out using anti-CD34 antibody, anti-CD43 antibody. The results are shown in FIG. 4. Since a sufficient number of cells could be confirmed in the CD34^{low}CD43⁺ cell fraction, it was confirmed that hematopoietic progenitor cells were induced.

C. Induction of T Cells from Hematopoietic Progenitor Cells.

Then, the obtained cells were seeded on OP9/DLL1 cells. In this step, cell sorting of the CD34^{low}CD43⁺ cell fraction was not performed. When this fraction is sorted, the efficiency of differentiation of T cells could be reduced in comparison with the case where sorting is not performed due to the decrease of the cells or damage to the cells by sorting.

Day 16: (Cells were Subcultured)

The cells loosely adhered to the OP9 cells were gently dissociated by pipetting several times. The cells were passed through a 100 μm mesh and collected in a 50 mL conical tube. The tube was centrifuged at 1203 rpm for 7 minutes at 4° C. The pellet was dispersed in 10 mL of medium B. Thus prepared cells were seeded on the OP9/DLL cells.

Day 23: (Cells were Subcultured) Blood Cell Colonies Began to Appear.

The cells loosely adhered to the OP9/DLL1 cells were gently dissociated by pipetting several times. The cells were passed through a 100 μm mesh and collected in a 50 mL conical tube. The tube was centrifuged at 1200 rpm for 7 minutes at 4° C. The pellet was dispersed in 10 mL of medium B.

Day 36: WT1 Tetramer Positive T Cells were Confirmed

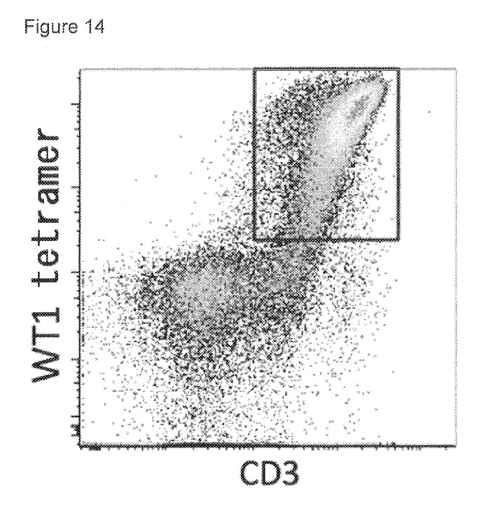

In order to confirm T cells specific for WT1 antigen were induced, the cells on Day 36 were analyzed by FACS with anti CD3 antibody and WT1 tetramer. Results are shown in FIG. 14. CD3′ cells were observed and a most part of the cells were differentiated into CD3′WT1 tetramer positive cells.

As shown above, the T cells regenerated from the T-iPS cells were confirmed to exhibit the same antigen specificity as the original T cells. Further, thus regenerated T cells expressed the surface antigen that were observed in mature T cells and therefore, had the well matured functions.

4) Antigen Specific Killer Activity of the T Cells Re-Generated from the WT1-T-iPS Cells.

1. CFSE labelled LCLs were used as target cells. The cells were suspended in the T cell medium and incubated in the presence of the WT1 peptide (SEQ ID NO: 2) for 2 hours.

2. The regenerated CD8 single positive T cells and the target cells (LCLs) were added to each well of a 96-well round bottom plate at different effector/target cell ratios of 0:1, 1:3, 1:1, 3:1 and 9:1. The cells were incubated in the presence of various concentrations of the peptide or absence of the peptide for 6 hours. After the incubation, the ratio of Annexin V positive cells to PI (Propidium Iodide) positive cells in the CFSE positive cell fraction were determined to confirm percentage of dead cells among the target cells.

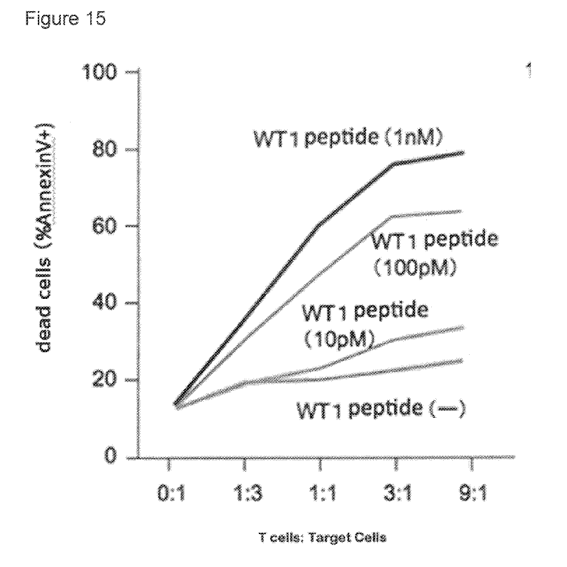

The regenerated WT1-CTL#9 showed high antigen specific cytotoxicity against the peptide-loaded LCLs (FIG. 15).

### Example 4

WT1 peptide specific CTL cells were induced according to the procedure of Example 3 from the same healthy volunteer from whom PBMC were obtained in Example 3. T-iPS cells (clone WT1#3-3) were established from the CTL and then, the T-iPS cells were differentiated into CD8 single positive T cells (re-generated WT1-CTL#3-3). The WT1 peptide used in Example 3 was also used in this example. The antigen specific killer activity of the re-generated CTLs against the LCLs loaded with the peptide as target cells was examined.

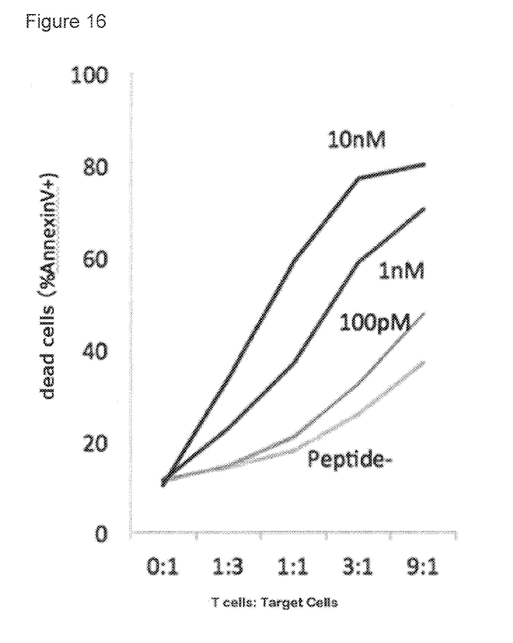

Results are shown in FIG. 16. The re-generated WT1-CTL#3-3 showed high antigen specific killing activity against the peptide-loaded LCLs.

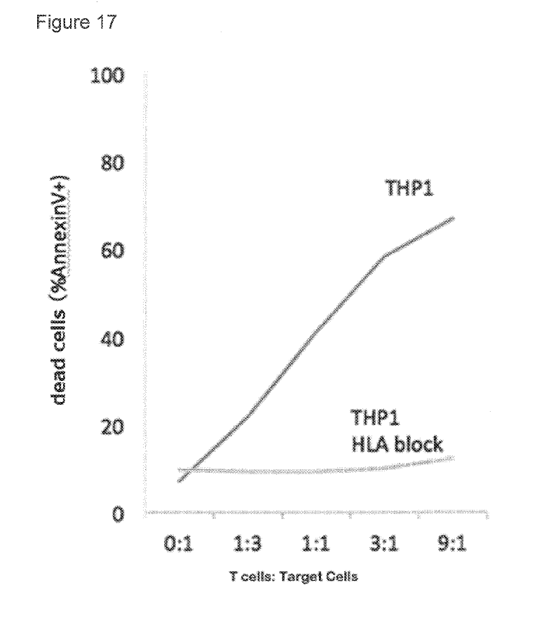

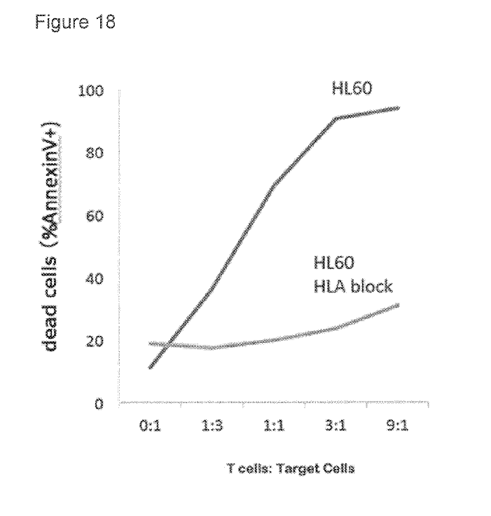

Cytotoxic activities of the re-generated WT1-CTL#3-3 against the leukemia cell lines THP1 and HL60 that expresses WT1 antigen were examined. In addition, whether or not anti-HLA class I antibody could block the cytotoxic activity was examined. The results are shown in FIGS. 17 and 18.

The re-generated WT1-CTLs#3-3 were cytotoxic against both cell lines THP-1 and HL60 that express WT1 antigen. The cytotoxic activities were completely blocked by the anti-HLA class I antigen. Based on the results, the re-generated WT1-CTL(#3-3) kill the leukemia cells in the antigen specific manner.

### Example 5

The allogenic reactivity of the regenerated CTLs against the peripheral blood monocytes of another person was examined. The cells used were as follows:

Effector cells: regenerated WT1-CTL#9 obtained in Example 3. Originated from a peripheral blood mononuclear cell of the volunteer having HLA-A*02:01/24:02 B*15:01/15:11; C*03:03/08:01; DRB1*12:01/12:02.

Target Cells: Peripheral blood mononuclear cells and B cells that were derived from the other volunteers.

Volunteer A: HLA-A*02:06/24:02; B*40:01/52:01; C*12:02/15:02; DRB1*08:02/15:02

Volunteer B: HLA-A*02:10/24:02; B*07:02/40:06; C*07:02/08:01; DRB1*04:05/04:05

The effector cells were fluorescently-labelled with CSFE. Peripheral blood monocytes and B cells obtained from the volunteers A and B were separately enriched from their peripheral blood by using anti DC14-MACS beads and anti CD19-MACS beads, respectively.

The degree of the cell division of the effector cells was determined by detecting the CFSE fluorescent intensity. When effector cells are activated, the division of the cells proceeds and the CFSE fluorescent intensity decreases.

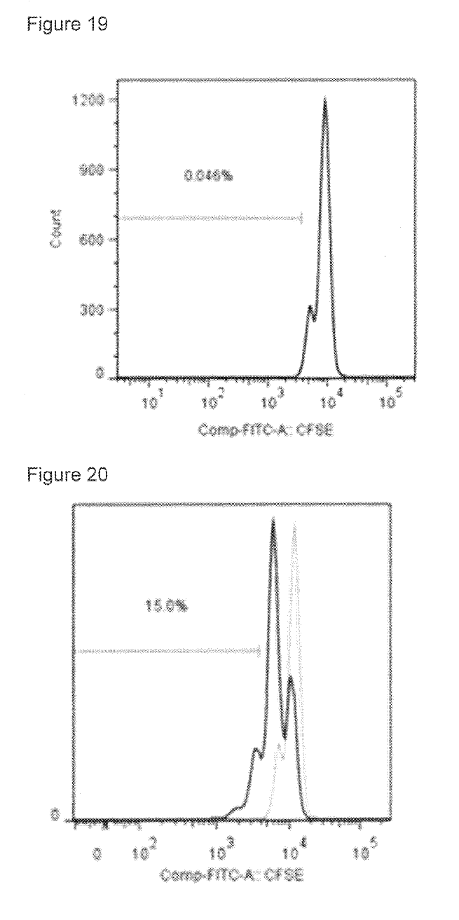

The effector cells or regenerated WT1-CTL#9 were cultured in a medium supplemented with only IL-7 (5 ng/mL) for 6 days without the target cells. The regenerated cells did not proliferate and the cells were not activated (Non-growth control, FIG. 19).

The regenerated CTLs were cultured in the presence of IL-2(20 U/mL), IL-7(5 ng/mL) and IL-15(10 ng/mL) without the target cells. Results are shown in FIG. 20. Compared with FIG. 19, the cells divided and proliferated a little. This data was used as control without the target cells and compared with the results obtained in the presence of the target cells.

The effector cells (8×10⁴ cells) and the target cells (2×10⁵ cells) were mixed and co-cultured for 6 days. Then, the fluorescent intensity of CFSE was measured to determine the degree of cell division.

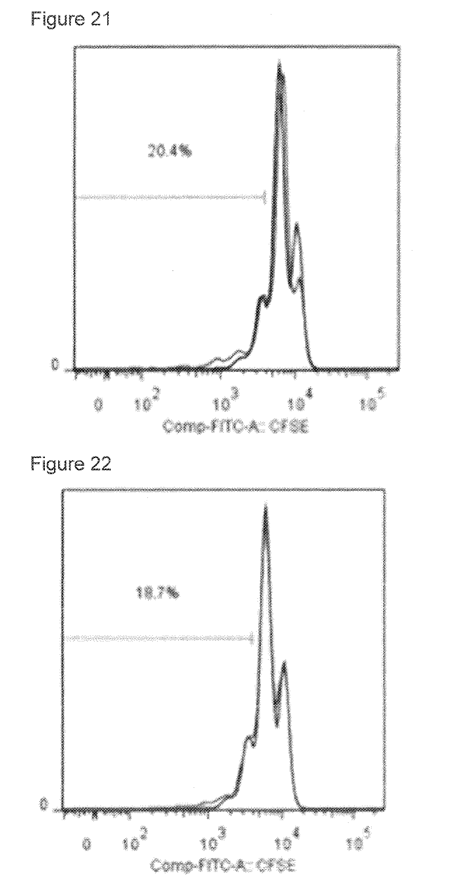

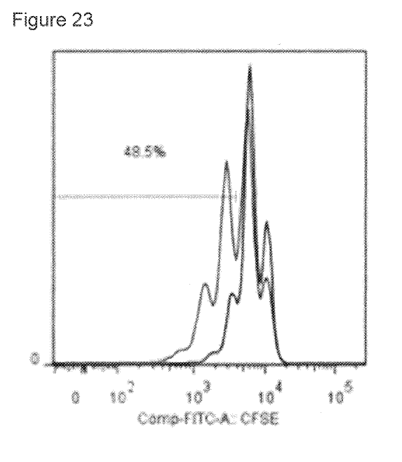

FIG. 21 shows the results obtained with peripheral blood monocytes and B cells both derived from the donor from which clone WT#9 was developed as target cells. The results were similar to that obtained without the target cells. That is, WT1-CTL#9 were not activated at all. The regenerated CTLs do not exert allogenic reactivity against the autologous HLAs.

FIG. 22 shows the results obtained with cells derived from peripheral blood of volunteer A as target cells. The regenerated CTL did not cause allogenic reaction against the target cells derived from volunteer A who had completely different HLAs. The regenerated WT1-CTL#9 cells were clonally expanded cells. It was confirmed that cloning of the T cells could avoid contamination of allogenically reactive T cells.

FIG. 22 shows the results obtained with cells derived from peripheral blood of volunteer B as target cells. The regenerated WT1-CTLs#9 were activated. That is, the cell-based immunotherapy in combination with the clone WT-CTL#9 and volunteer B is dangerous. Even if the T cells were clonally expanded, the risk of exerting allogenic reaction cannot avoid completely. Accordingly, upon conducting the cell-based immunotherapy, the clonally expanded regenerated CTL clone must be screened for safety before administering the CTLs to the patient.

1. A method for inducing T cells for a cell-based immunotherapy, which comprises the steps of:
(1) providing human pluripotent stem cells bearing a T cell receptor specific for a desired antigen, and
(2) inducing T cell progenitors or mature T cells from the pluripotent stem cells of step (1).

2. The method according to claim 1, wherein the human pluripotent stem cells bearing a T cell receptor specific for a desired antigen is obtained by inducing pluripotent stem cells from a human T cell specific for the desired antigen.

3. The method according to claim 2, wherein the human T cell specific for the desired antigen is obtained from a person who is not the subject to be treated by the cell-based immunotherapy, and is suffered from or had previously been suffered from the disease to be treated by the cell-based immunotherapy.

4. The method according to claim 2, wherein the human T cell specific for the desired antigen is obtained from a person who is not the subject to be treated by the cell-based immunotherapy, and has never been suffered from the disease to be treated by the cell-based immunotherapy.

5. The method according to claim 2, wherein the human T cell specific for the desired antigen is a T cell of a person having HLA phenotypes that are completely or partially identical to the HLA phenotypes of the subject to be treated by the cell-based immunotherapy.

6. The method according to claim 4, wherein the human T cell specific for the desired antigen is a T cell of a person homozygous for HLA haplotype that matches at least one of HLA haplotypes of the subject to be treated.

7. The method according to claim 1, further comprises the step of co-culturing the T cell progenitors or mature T cells induced from the pluripotent stem cells with the lymphocytes of the subject to be treated by the cell based immunotherapy to verify that the T cells are not allogenicaly reactive against the subject.

8. The method according to claim 1, wherein the human pluripotent stem cells are human iPS cells.

9. The method according to claim 1, wherein the cell-based immunotherapy is for the treatment of a disease selected from the group consisting of a cancer, an infectious disease, an autoimmune disease and an allergy.

10. The method according to claim 9, wherein the cancer is an EB virus relating cancer.

11. The method according to claim 9, wherein the infectious disease is an EB virus associated disease.

12. The method according to claim 9, wherein the cancer expresses WT1 gene.


SEQUENCE LISTING (2 sequences)

SEQ ID NO: 1 - protein, 9 aa, Homo sapiens
IYVLVMLVL

SEQ ID NO: 2 - protein, 9 aa, Homo sapiens
IYVLVMLVL